(19) United States
(12) Patent Application Publication
(10) Pub. No.: US 2009/0287221 A1
(43) Pub. Date: Nov. 19, 2009

(54) Tissue Modification Device and Methods of Using the Same

(75) Inventors: Paul Sand, San Carlos, CA (US); Bryce Way, San Jose, CA (US); Murray David Solsberg, Englewood, CO (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Vertos Medical, Inc.

(21) Appl. No.: 12/466,269

(22) Filed: May 14, 2009

Related U.S. Application Data
(60) Provisional application No. 61/053,075, filed on May 14, 2008.

(51) Int. Cl. A61B 17/26; A61B 17/00

(52) U.S. Cl. 606/110; 606/205

(57) ABSTRACT

The present disclosure is directed to a device for excising tissue including an outer hollow member having a lumen communicating with a distal opening. The device also includes an elongate member having a longitudinal axis and a distal end, the elongate member being longitudinally movable within the outer hollow member lumen such that the distal end of the elongate member can be moved from a first configuration distal to the outer hollow member distal opening and a second configuration more proximal to the outer hollow member distal opening. Further, the device includes at least one grasping member hingedly disposed about a distal portion of the elongate member along a generally longitudinal axis, wherein movement of the elongate member from the first configuration to the second configuration can cause displacement of the at least one grasping member about the generally longitudinal axis.

This application claims priority under 35 U.S.C. § 119 to U.S. Provisional Patent Application No. 61/053,075, filed May 14, 2008, which is herein incorporated by reference in its entirety.

### FIELD OF THE INVENTION

This invention relates generally to the field of minimally invasive surgery. More specifically, the invention relates to minimally invasive methods and devices for modifying and/or extracting tissue.

### BACKGROUND OF THE INVENTION

The vertebral column (spine, spinal column, backbone) forms the main part of the axial skeleton, provides a strong yet flexible support for the head and body, and protects the spinal cord disposed in the vertebral canal, which is formed within the vertebral column. The vertebral column comprises a stack of vertebrae with an intervertebral disc between adjacent vertebrae. The vertebrae are stabilized by muscles and ligaments that hold the vertebrae in place and limit the movements of the vertebrae.

As illustrated in FIG. 1, each vertebra 10 includes a vertebral body 12 that supports a vertebral arch 14. A median plane 210 generally divides vertebra 10 into two substantially equal lateral sides. Vertical body 12 has the general shape of a short cylinder and is anterior to the vertebral arch 14. The vertebral arch 14 together with vertebral body 12 encloses a space termed the vertebral foramen 15. The succession of vertebral foramen 15 in adjacent vertebrae 10 along the vertebral column define the vertebral canal (spinal canal), which contains the spinal cord.

Vertebral arch 14 is formed by two pedicles 24 which project posteriorly to meet two laminae 16. The two laminae 16 meet posteriomedially to form the spinous process 18. At the junction of pedicles 24 and laminae 16, six processes arise. Two transverse processes 20 project posterolaterally, two superior articular processes 22 project generally superiorly and are positioned superior to two inferior articular processes 25 that generally project inferiorly.

The vertebral foramen 15 is generally an oval shaped space that contains and protects the spinal cord 28. Spinal cord 28 comprises a plurality of nerves 34 surrounded by cerebrospinal fluid (CSF) and an outermost sheath/membrane called the dural sac 32. The CSF filled dural sac 32 containing nerves 34 is relatively compressible. Posterior to the spinal cord 28 within vertebral foramen 15 is the ligamentum flavum 26. Laminae 16 of adjacent vertebral arches 14 in the vertebral column are joined by the relatively broad, elastic ligamentum flavum 26.

In degenerative conditions of the spine, narrowing of the spinal canal (stenosis) can occur. Lumbar spinal stenosis is often defined as a dural sac cross-sectional area less than 100 mm² or an anterior-posterior (AP) dimension of the canal of less than 10-12 mm for an average male.

The source of many cases of lumbar spinal stenosis is thickening of the ligamentum flavum. Spinal stenosis may also be caused by subluxation, facet joint hypertrophy, osteophyte formation, underdevelopment of spinal canal, spondylosis deformans, degenerative intervertebral discs, degenerative spondylolisthesis, degenerative arthritis, ossification of the vertebral accessory ligaments and the like. A less common cause of spinal stenosis, which usually affects patients with morbid obesity or patients on oral corticosteroids, is excess fat in the epidural space. The excessive epidural fat compresses the dural sac, nerve roots and blood vessels contained therein and resulting in back, leg pain and weakness and numbness of the legs. Spinal stenosis may also affect the cervical and, less detached from the spine during the laminectomy, these patients frequently develop spinal instability post-operatively.

Patients suffering from spinal stenosis are typically first treated with exercise therapy, analgesics, and anti-inflammatory medications. These conservative treatment options frequently fail. If symptoms are severe, surgery is required to decompress the spinal cord and nerve roots.

In some conventional approaches to correct stenosis in the lumbar region, an incision is made in the back and the muscles and supporting structures are stripped away from the spine, exposing the posterior aspect of the vertebral column. The thickened ligamentum flavum is then exposed by removal of a portion of the vertebral arch, often at the laminae, covering the back of the spinal canal (laminectomy). The thickened ligamentum flavum ligament can then be excised by sharp dissection with a scalpel or punching instruments such as a Kerison punch that is used to remove small chips of tissue. The procedure is performed under general anesthesia. Patients are usually admitted to the hospital for approximately five to seven days depending on the age and overall condition of the patient. Patients usually require between six weeks and three months to recover from the procedure. Further, many patients need extended therapy at a rehabilitation facility to regain enough mobility to live independently.

Much of the pain and disability after an open laminectomy results from the tearing and cutting of the back muscles, blood vessels, supporting ligaments, and nerves that occurs during the exposure of the spinal column. Also, because the spine stabilizing back muscles and ligaments are stripped and detached from the spine during the laminectomy, these patients frequently develop spinal instability post-operatively.

Minimally invasive techniques offer the potential for less post-operative pain and faster recovery compared to traditional open surgery. Percutaneous interventional spinal procedures can be performed with local anesthesia, thereby sparing the patient the risks and recovery time required with general anesthesia. In addition, there is less damage to the paraspinal muscles and ligaments with minimally invasive techniques, thereby reducing pain and preserving these important stabilizing structures.

Various techniques for minimally invasive treatment of the spine are known. Microdiscectomy is performed by making a small incision in the skin and deep tissues to create a portal to the spine. A microscope is then used to aid in the dissection of the adjacent structures prior to discectomy. The recovery for this procedure is much shorter than traditional open discectomies. Percutaneous discectomy devices with fluoroscopic guidance have been used successfully to treat disorders of the disc but not to treat spinal stenosis or the ligamentum flavum directly. Arthroscopy or direct visualization of the spinal structures using a catheter or optical system have also been proposed to treat disorders of the spine including spinal stenosis, however these devices still use miniaturized standard surgical instruments and direct visualization of the spine similar to open surgical procedures. These devices and techniques are limited by the small size of the canal and these operations are difficult to perform and master. In addition, these procedures are painful and often require general anesthesia. Further, the arthroscopy procedures are time consuming and the fiber optic systems are expensive to purchase and maintain.

Still further, because the nerves of the spinal cord pass through the spinal canal directly adjacent to and anterior to the ligamentum flavum, any surgery, regardless of whether open or percutaneous, includes a risk of damage to the nerves of the spinal cord.

Hence, it remains desirable to provide simple methods, techniques, and devices for treating spinal stenosis and other spinal disorders without requiring open surgery. It is further desired to provide a system whereby the risk of damage to the dural sac containing the spinal nerves may be reduced.

### BRIEF DESCRIPTION OF THE DRAWINGS

For a detailed description of the preferred embodiments of the invention, reference will now be made to the accompanying drawings in which:

FIG. 1 is cross-sectional view of the spine viewed from the space between two vertebrae, showing the upper surface of one vertebra and the spinal canal with the dural sac and a normal (unstenosed) ligamentum flavum therein;

FIGS. 2A-D are perspective views of four embodiments of a device for modifying and/or excising tissue;

FIGS. 3A-E are distal end views of alternative embodiments of grasping members that may be employed in the devices of FIGS. 2A-D;

FIG. 4 is a perspective view of an embodiment of a device for modifying and/or excising tissue; and

FIGS. 5A-D are select schematic views of an embodiment of a method of excising tissue using the device of FIGS. 2A-D.

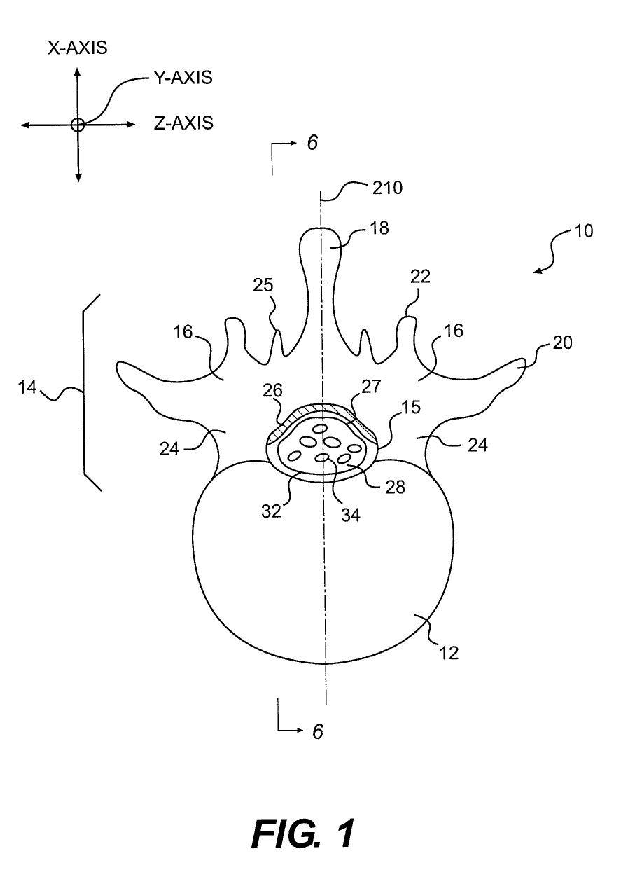

### DESCRIPTION OF THE EMBODIMENTS

Embodiments of tissue modifying and excision devices and methods disclosed herein may take several forms and may be employed as a part of the Ipsilateral Approach to Minimally Invasive Ligament Decompression Procedure (ILAMP) described in U.S. patent application Ser. No. 11/382,349, which is hereby incorporated herein by reference in its entirety, as a part of the Minimally Invasive Ligament Decompression Procedure (MILD) described in U.S. patent application Ser. No. 11/193,581, which is hereby incorporated herein by reference in its entirety, or used according to any other suitable procedure.

In the descriptions of the embodiments of tissue modifying and excision devices and methods of using the same below, the distal portions of the devices are described in detail. As used herein, the term “distal” refers to positions that are relatively closer to the region of interest (e.g., the thickened portion of the ligamentum flavum to be decompressed), whereas the term “proximal” refers to positions that are relatively further from the region of interest.

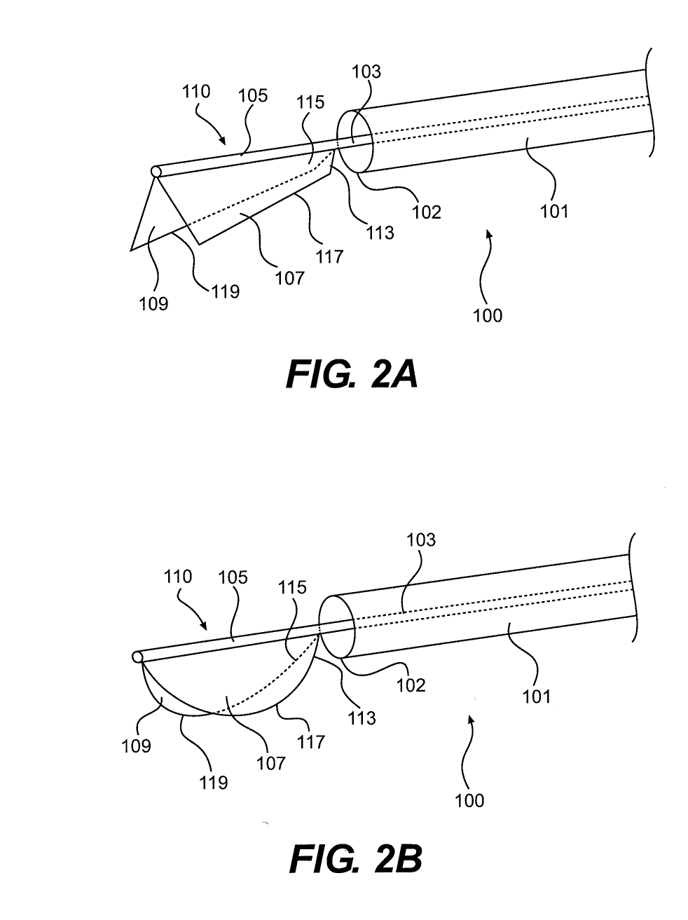

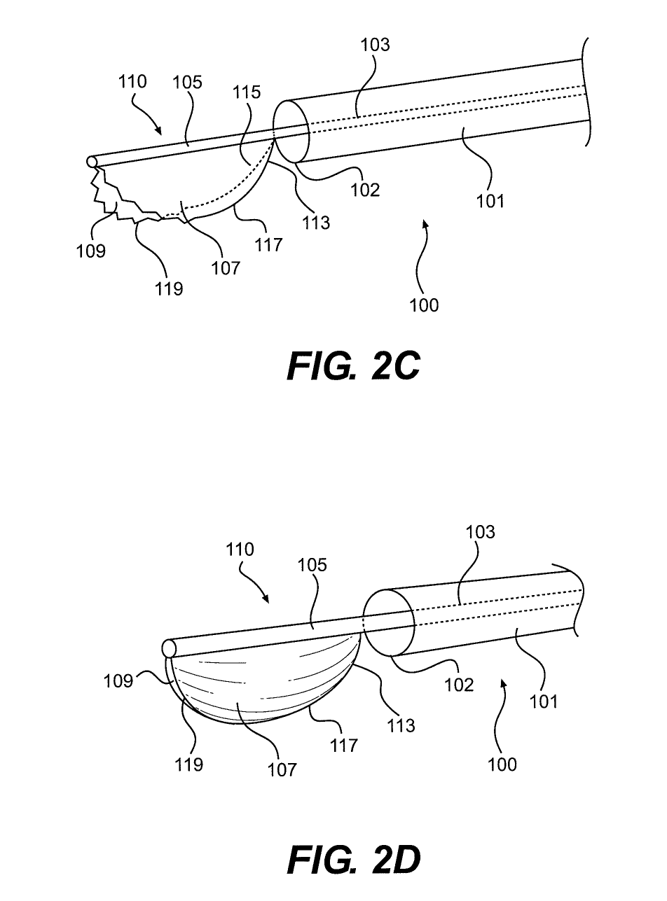

Referring now to FIGS. 2A-D, embodiments of a minimally invasive tissue modifying device 100 are shown. Device 100 generally includes an outer tubular member 101 and an inner elongate member 103 at least partially disposed within outer tubular member 101. Outer member 101 can include a distally located aperture 102. Device 100 can also include a first grasping member 107 and a second grasping member 109 disposed about a generally longitudinal axis of a distal portion 110 of inner elongate member 103.

Outer hollow member 101 can have a lumen communicating with distal opening 102. Elongate member 103 can have a longitudinal axis and a distal end. Further, elongate member 103 can be longitudinally movable within the outer hollow member lumen such that the distal end of elongate member 103 can be moved from a first configuration distal to distal opening 102 and a second configuration more proximal to distal opening 102.

Device 100 can also include at least one grasping member hingedly disposed about a distal portion of elongate member 103 along a generally longitudinal axis, wherein movement of elongate member 103 from the first configuration to the second configuration can cause displacement of the at least one grasping member about the generally longitudinal axis. Thus, grasping members 107, 109 may be generally described as having at least a generally expanded or open position and a collapsed or closed position.

In some embodiments, first and second grasping members 107, 109 may generally rotate about a hinged connection 105. In other embodiments, each grasping member may be associated with different hinges. In operation, first and second grasping members 107, 109 may be configured such that when elongate member 103 is at least partially retracted via longitudinal movement into the interior of outer tubular member 101, grasping members 107, 109 may at least partially collapse and fold together to compress, capture, modify, or cut tissue generally disposed between members 107, 109. In some embodiments, only one grasping member may move.

Outer tubular member 101 can be generally cylindrical and may include any suitable medical device. Examples include, without limitation, cannulas, catheters, hypotubes, and the like.

Inner elongate member 103 may include any suitable geometry, such as, without limitation, a rod or a tube. Generally, inner elongate member 103 has an outer diameter that is less than an inner diameter of outer member 101 such that member 103 may be disposed within outer member 101. In some instances, the distal tip of inner elongate member 103 may be sharpened to form a tip for ease of insertion into tissue.

As shown in FIG. 2A, first and second grasping members 107, 109 are hingedly associated with elongate member 103 and intersect to form an acute angle in their expanded position. Grasping members 107, 109 may form any suitable angle in their expanded position. Hinged connection 105 may be biased in an expanded or open position. For example, hinged connection 105 may include a spring mechanism such that, unless force is applied to grasping members 107, 109, the grasping members 107, 109 remain in their expanded position. As a result, when grasping members 107, 109 are extended from outer tubular member 101, they may transition to an open position.

As mentioned above, grasping members 107, 109 may at least partially shut or collapse to enclose tissue when moved longitudinally within outer member 101. In a closed or collapsed position, grasping members 107, 109 can be configured to fit within outer tubular member 101. In other embodiments, grasping members 107, 109 may only at least partially collapse or close, and/or partially reside within outer member 101. In some embodiments, grasping member 107 may move while grasping member 109 may remain stationary.

At least one grasping member 107, 109 may be configured to deflect. In particular, a grasping member may be configured to deflect about an axis generally longitudinal to the longitudinal axis of inner member 103. Similar to the rotational movement of a grasping member, a deflection of a grasping member may be actuated via longitudinal movement of inner member 103 relative to outer member 101. In some embodiments, a grasping member may be configured for both rotation and deflection. Also, grasping member 107 may be rotated while grasping member 109 may be deflected.

Grasping members 107, 109 may have any suitable geometrical shape. In some embodiments, the shape of the grasping members 107, 109 may allow grasping members 107, 109 to move toward each other by a camming action. For example, longitudinal movement of inner elongate member 102 may engage a proximal edge of a grasping member with a distally located feature of outer tubular member 101. In particular, outer edges 117, 119 of grasping members 107, 109 can be configured such that the proximal portions 113, 115 of outer edges 117, 119 may engage aperture 102 of outer tubular member 101 and cam towards each other as grasping members 107, 109 are longitudinally retracted into outer tubular member 101.

As an example, FIG. 2A depicts a trapezoidal shape of the grasping members 107, 109. FIG. 2B shows grasping members 107, 109 with a curved or semi-circular geometry. In another embodiment, grasping members 107, 109 may have a triangular geometry (not shown). FIG. 2C shows part of outer edges 117, 119 with serrations. FIG. 2D shows grasping members 107, 109 with a concave, or scooped surface, configured to engage tissue. Such a “clam-shell” embodiment may permit tissue to be substantially surrounded by grasping member 107, 109. Although shown with two similar grasping members, grasping members 107, 109 can be different. For example, grasping member 107 may be trapezoidal while grasping member 109 may be curved.

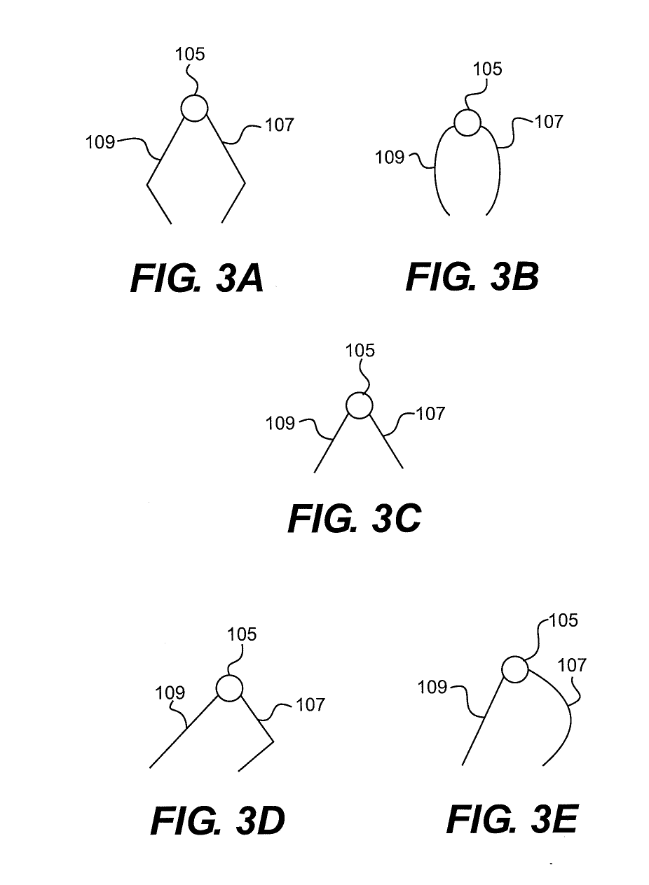

FIGS. 3A-E illustrate cross-sectional views of some alternative embodiments of grasping members 107, 109. Grasping members 107, 109 may be configured for excision or extraction of specific tissue, such as, for example, ligamentum flavum. As shown in FIG. 3A, grasping members 107, 109 may be bent, or curved as shown in FIG. 3B. In some embodiments, grasping members 107, 109 may be planar as shown in FIG. 3C. In other embodiments, grasping members 107, 109 may be different, as shown in FIGS. 3D and 3E.

Some edges of grasping members 107, 109 may be at least partially beveled or sharpened. Also, some edges may be at least partially toothed or jagged so as to further facilitate excision of tissue, as shown in FIG. 2C. Further, inner surfaces of grasping members 107, 109 may be textured to enhance the grasping and extraction of tissue. In particular, part of the inner surfaces of grasping members 107, 109 may have a plurality of protrusions or spikes to better grasp tissue. In other embodiments, some edges of grasping members 107, 109 may have cutting edges configured to cut tissue. For example, grasping members 107, 109 may be arranged such that the cutting edges overlap when grasping members 107, 109 close. Various types of edges may further facilitate removal or excision of tissue. Any part of an edge of a grasping member can include one or more of the features outlined above.

The components of device 100 may comprise any suitable material(s) including without limitation metals (e.g., stainless steel, titanium, etc.), non-metals (e.g., polymer, composites, etc.), or combinations thereof. The components of device 100 can be manufactured from a durable biocompatible material such as titanium or stainless steel, and may be polymeric. In particular, the grasping members (e.g., grasping members 107, 109) can include a biocompatible resilient material capable of rotating and/or flexing from the generally open position to a partially closed position. Such a material may permit grasping members to spring back to a more open position when extended from outer tubular member 101.

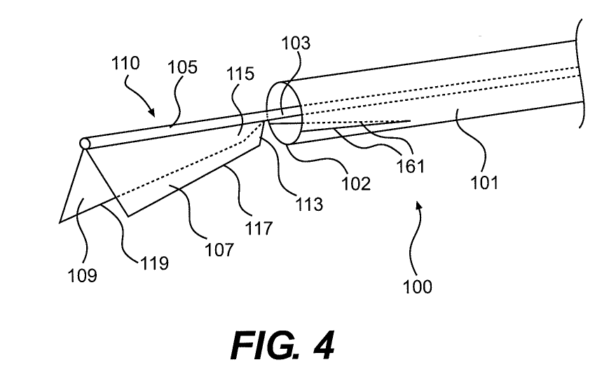

FIG. 4 shows another embodiment of device 100 whereby grasping members 107, 109 may be longitudinally moved to transition to a more closed position by the actuation of one or more slots 161 in outer tubular member 101. Slots 161 may be positioned and oriented to at least partially form a cam upon which grasping members 107, 109 may contact. Such contact may cause one or more grasping members into a partially closed position as grasping members 107, 109 are moved proximally. For example, one or more slots 161 may be angled toward each other, or curved.

In such embodiments, the proximal ends of grasping members 107, 109 can be aligned to engage with slots 161 when grasping members 107, 109 are in a generally open position. As grasping members 107, 109 are retracted longitudinally into tubular member 101, grasping members 107, 109 may slide into slots 161. The configuration of slots 161 can force grasping members 107, 109 into a partially closed position via a rotational, deflectional, or combination of movement. Accordingly, in such an embodiment, slots 161 may actuate grasping members 107, 109. In some embodiments, a combination of grasping member geometry and slots 161 may be used to at least partially move one or more grasping members 107, 109 upon longitudinal movement of inner member 103 relative to outer member 101.

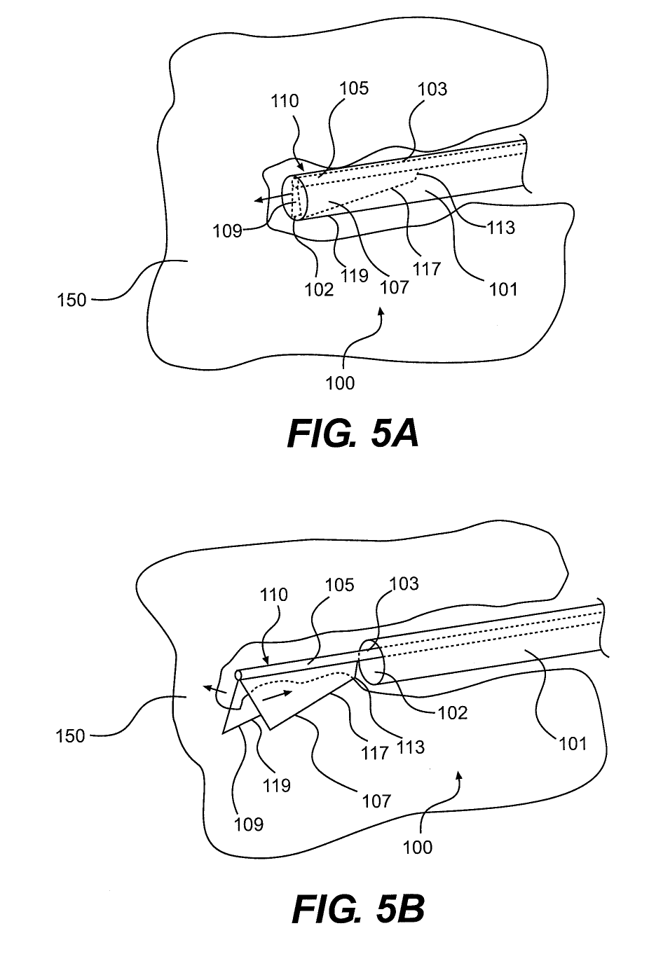

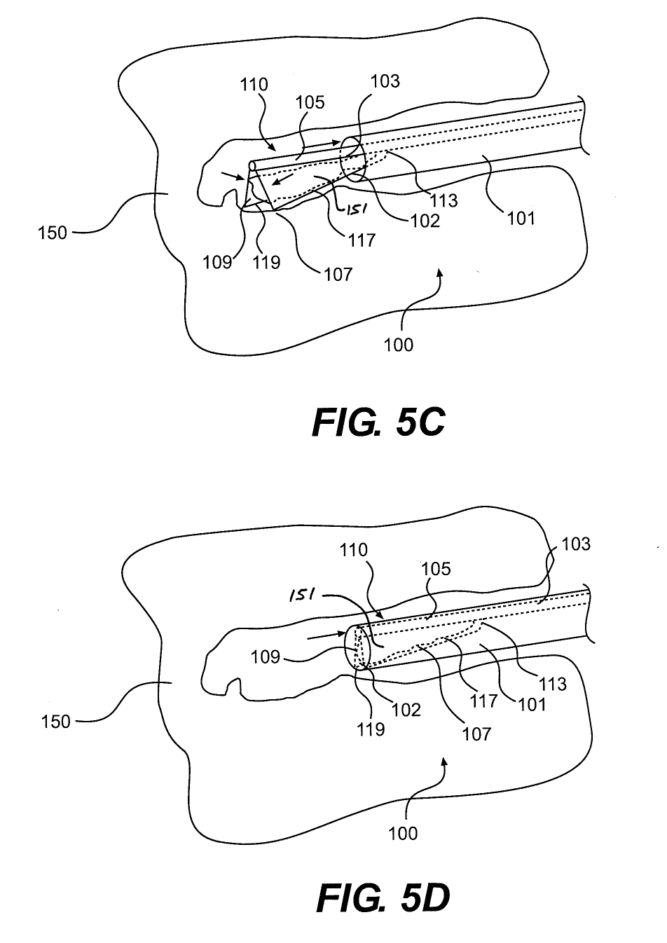

FIGS. 5A-D illustrate the operation of device 100. Operation of the various embodiments of the device 100 described above can be similar.

In operation, device 100 may be inserted at the desired site 150 of tissue excision, as shown in FIG. 5A. Insertion may be percutaneous, and may include accessing the epidural space of the spinal canal. Device 100 may be inserted with either distal end of inner elongate member 103 extended from outer tubular member 101 or with member 103 at least partially residing inside outer tubular member 101. Once the distal end of device 100 has been inserted at the target excision site 150, distal end of elongate member 103 may be extended longitudinally. Grasping members 107, 109 may move laterally outward to at least partially assume an open or expanded position, as shown in FIG. 5B.

Once one or more grasping members 107, 109 are at least partially expanded, one or more grasping members 107, 109 may engage the tissue to be excised. This tissue could include ligamentum flavum. Device 100 can be positioned such that the tissue to be modified and/or excised is generally disposed between grasping members 107, 109. Then, inner elongate member 103, and hence grasping members 107, 109, may be longitudinally moved in a generally proximal direction. As elongate member 103 is longitudinally moved into outer tubular member 101, grasping members 107, 109 can at least partially move toward each other, thereby engaging the tissue 151 disposed between grasping members 107, 109, as shown in FIG. 5C. As shown in FIG. 5C, grasping members 107, 109 may deflect about tissue 151 to permit retention of tissue 151 by grasping members 107, 109. In other embodiments, such as, for example, as shown in FIG. 2D, grasping members 107, 109 having a clam-shell design may engulf tissue 151 with little or no deflection. Tissue 151 generally within grasping members 107, 109 may then be removed from the site via outer tubular member 101, as shown in FIG. 5D. The above described method may be repeated multiple times to remove or excise tissue from site 150.

While the embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

What is claimed is:

1. A device for excising tissue comprising:
an outer hollow member having a lumen communicating with a distal opening;
an elongate member having a longitudinal axis and a distal end, the elongate member being longitudinally movable within the outer hollow member lumen such that the distal end of the elongate member can be moved from a first configuration distal to the outer hollow member distal opening and a second configuration more proximal to the outer hollow member distal opening; and
at least one grasping member hingedly disposed about a distal portion of the elongate member along a generally longitudinal axis, wherein movement of the elongate member from the first configuration to the second configuration causes displacement of the at least one grasping member about the generally longitudinal axis.

2. The device of claim 1, wherein the at least one grasping member further comprises a first grasping member and a second grasping member, and wherein movement of the elongate member from the first configuration to the second configuration causes displacement of the first and second grasping members toward each other.

3. The device of claim 2, wherein the first and second grasping members are hingedly disposed about the elongate member at different locations.

4. The device of claim 1, wherein the at least one grasping member has a tissue engagement surface that is generally concave.

5. The device of claim 1, wherein the at least one grasping member includes an engaging edge configured to contact the distal opening of the outer member to cause displacement of the at least one grasping member.

6. The device of claim 1, wherein the outer member has at least one slot configured to at least partially engage the at least one grasping member to cause displacement of the at least one grasping member.

7. The device of claim 1, wherein the at least one grasping member includes at least one of a linear edge, a bent edge, a curved edge, a cutting edge, and a serrated edge.

8. A device for excising tissue comprising:
an outer hollow member having a distal opening;
an inner elongate member having a distal portion and being longitudinally movable within the outer hollow member; and
at least one grasping member disposed about a generally longitudinal axis of the distal portion of the inner member, wherein longitudinal movement of the inner member relative to the outer member causes deflection of the at least one grasping member.

9. The device of claim 8, wherein the device includes a first grasping member and a second grasping member and longitudinal movement of the inner member relative to the outer member causes the first and second grasping members to move toward each other.

10. The device of claim 8, wherein the at least one grasping member has a concave surface configured to engage tissue.

11. The device of claim 8, wherein the at least one grasping member includes an engaging edge configured to contact the distal opening of the outer member to actuate deflection of the at least one grasping member.

12. The device of claim 8, wherein outer member has at least one slot configured to at least partially engage the at least one grasping member to actuate deflection of the at least one grasping member.

13. The device of claim 8, wherein the at least one grasping member includes at least one of a linear edge, a bent edge, a curved edge, a cutting edge, and a serrated edge.

14. The device of claim 8, wherein the at least one grasping member is configured for rotational movement about a generally longitudinal axis of the inner member.

15. A method of excising tissue, comprising:
providing an elongated tissue excision device having at least one-grasping member actuated by longitudinal movement;
contacting the at least one grasping member with the tissue to be excised; and
longitudinally actuating the at least one grasping member to at least partially engage the tissue by rotation or deflection of the at least one grasping member about a generally longitudinal axis.

16. The method of claim 15, wherein the tissue includes a ligamentum flavum.

17. The method of claim 16, further including excising at least a portion of the ligamentum flavum.

18. The method of claim 15, further including percutaneously accessing the epidural space.

19. The method of claim 15, further including compressing the dural sac within the spinal canal.

20. The method of claim 15, wherein the tissue excision device includes an outer hollow member, an inner elongate member longitudinally movable within the outer hollow member, and wherein the at least one grasping member is disposed about a generally longitudinal axis of the inner member.
